(19) United States
(12) Patent Application Publication
(10) Pub. No.: US 2012/0255502 A1
(43) Pub. Date: Oct. 11, 2012

(54) PACKAGE AND METHOD OF MARKETING

(75) Inventor: Robert HOLMES, North Shore City (NZ)

(73) Assignee: ALLEVA ANIMAL HEALTH LIMITED, Auckland (NZ)

(21) Appl. No.: 13/433,671

(22) Filed: Mar. 29, 2012

(30) Foreign Application Priority Data
Apr. 11, 2011 (NZ) 592175

(51) Int. Cl. A61D 7/00; A61K 31/365; A61K 31/4439; A61K 31/216; A61K 31/22; A61K 31/09; A61K 31/7048; A61K 31/341; A61K 31/155; A61K 31/5377; A61K 31/4402; A61K 31/4985; A61P 33/00; A61P 33/14; B65D 85/00; B65D 75/36; A61K 31/415

(52) U.S. Cl. 119/651; 514/407; 514/453; 514/341; 514/531; 514/549; 514/721; 514/30; 514/471; 514/637; 514/28; 514/232.2; 514/345; 514/250; 206/571; 206/572; 206/570

(57) ABSTRACT

A method of treatment and a package that is designed to accomplish this. The package provides the pet owner with a pack containing individual doses of topically applied medications pre-packaged with an oral taste treat. The topically applied medication contains at least one pharmaceutically active ingredient selected from the group of chemical families comprising phenylpyrazoles, macrocyclic lactones, neonicotinoids, synthetic pyrethroids, insect growth regulators, amidines, semicarbazones, spinosyns, octadepsipeptides, and pyrazinoisoquinolines, or any combination thereof. The treat can be administered either before the topically applied treatment is given so as to relax and distract the pet, or after the treatment is given so as to reward the pet and help it forget the stress of treatment. The treat can also be shaped to signify the type of animal to be treated.

### FIELD OF THE INVENTION

The present invention relates to a package for topically treating a pet with a medication or shampoo while also ameliorating the negative emotional aspects associated with the treatment. It has particular application to a method of marketing and also to methods of treatment of animals.

### BACKGROUND OF THE INVENTION

There are many treatments for pet that involve a measure of physical or emotional discomfort for either the pet or the pet owner. For example the application of topical flea treatments requires the owner to hold the pet firmly to prevent it from struggling while applying a solvent based liquid to a spot on the back of the animal. This is a procedure that can cause the owner to suffer from feelings of anguish with a corresponding feeling of resentment toward the owner by the pet.

For many people who highly value the bond they have with their pet this situation is particularly undesirable. Because while they would consider the need to give such treatments very necessary, they are also probably left with feelings of guilt from having made their pet suffer through an unpleasant ordeal.

Typically the manufacturers of such topical treatments quite rightly focus attention on making their formulations as easy to apply and as non-hazardous to the pet as possible. This helps to eliminate the most significant negative physical aspects of the treatment occasion. However removing the negative physical aspects from the treatment itself does not address entirely the negative emotional aspects the treatment occasion may have on both human and pet nor does it address the damage it may do to the bond between them.

As societal living patterns have changed family size has reduced considerably. In western societies it is no longer common for families to live in extended groups. Living situations comprising a single family or just one or two people are now the norm. In such an environment it has become very common for people to own a cat or dog or other non-human companion. These pets then become an important and central aspect of the life of the owner. They provide companionship and a sense of dependence.

Among the other benefits of owning a pet are that they encourage exercise. Pets can provide comfort. The simple act of petting an animal can help a person relax. Pet ownership may even contribute to lower blood pressure, heart rate, and cholesterol.

Over time the owner will often form a deep sense of emotional dependence on their pet. Given the nature of such a relationship the pet owner will do all they can to enhance this mutual bond between themselves and their pet. For example; many owners will celebrate the birthday of their pet or will spend many thousands of dollars on their physical care and well-being. Pet owners also spend a considerable portion of their income on the nutritional needs of their pets.

However there are times when this mutual bond is weakened. Such as when the owner travels, is physically separated from the pet for a period of time, or when the pet suffers an illness and must visit a vet. On such occasions the pet probably feels a sense of loss and betrayal while the owner feels a sense of guilt or anguish.

Another occasion arises when the pet is subjected to a degree of discomfort in the presence of the owner is when they undergo an unpleasant veterinary medication treatment at home, such as administering oral tablets. On these occasions the pet owner will feel guilty they have administered a treatment that while beneficial has bought a degree of physical discomfort to the pet. The pet on the other hand will also no doubt feel resentful that they have been subjected to this degree of discomfort, particularly when they will have no comprehension that it was meant to be beneficial to them in some way.

It is generally accepted that treating a pet with a topical medication or shampoo is less problematic than treating them with an oral medication. This is to a large extent true but there still remain several negative aspects to such a treatment occasion from both the viewpoint of the owner and from the viewpoint of the pet.

The Owner must seize the pet and hold them tightly while applying the tip of the medication applicator against the skin. If the pet is not used to such a treatment it is likely to struggle and scratch or attack the owner. After the treatment the pet may also spend time trying to lick or rub the medication off.

Pets that have previously had treatment with a topical treatment may also develop an aversion to future treatments. Aversive behavior may be triggered by the odor of the medication or by the sight and sound of the pet owner unwrapping and preparing to administer the treatment.

What is needed is a method of minimizing or ameliorating the negative emotional aspects associated with the application of topically administered medications or shampoos.

### OBJECT OF THE INVENTION

It is an object of the invention to provide a package for the treatment of pet animals or a method of treatment of pet animals making use of such a package that ameliorates some of the disadvantages and limitations of the known art, or at least provides the public with a useful choice.

### SUMMARY OF INVENTION

In a first aspect the invention provides a veterinary kit containing at least one individual dose of a topical treatment and least one orally administered taste treat, with said topical treatment selected from, but not limited to, topical medicines, topical ointments, and topical shampoos.

In another aspect the invention provides a blister pack for the treatment of a pet, the blister pack containing a plurality of individual dosages of a topical treatment (such as a topical medication or shampoo) each dosage suitable for topical administration to a pet animal and including in the same blister pack a number of oral treats for a pet animal in close proximity to the individual topical treatment dosages.

In a second aspect the invention provides a method of treatment of a pet animal with a topical medicine or shampoo by providing the pet owner with a package that contains individual doses of a topical treatment (such as a topical medication or shampoo) pre-packaged with an oral taste treat, applying the topical treatment to the pet animal, and removing the oral taste treat from the package and administering it to the pet animal either (a) before the topically applied treatment is given so as to relax the pet, or (b) during or after the treatment is given so as to reward the pet and help it forget the negative aspects of the treatment.

Preferably the treatment is a topically applied medication, such as a spot on to be applied to a dog or a cat.

In another aspect the invention provides a veterinary kit containing at least one individual dose of a topical treatment and at least one orally administered taste treat, with said topical treatment comprising a liquid spot-on treatment for cats or dogs administered for the purpose of ectoparasite and/or endoparasite control, and in which the topical treatment contains at least one pharmaceutically active ingredient selected from the group of chemical families comprising phenylpyrazoles, macrocyclic lactones, neonicotinoids, synthetic pyrethroids, insect growth regulators, amidines, semicarbazones, spinosyns, octadepsipeptides, and pyrazinoisoquinolines; or any combination thereof.

By way of example these chemical families include (but are not limited to) the following known actives:

Phenylpyrazoles=fipronil, pyriprole
Macrocyclic lactones=moxidectin, selamectin
Neonicotinoids=imidacloprid, dinotefuran
Synthetic pyrethroids=permethrin, cyphenothrin, etofenprox,
Insect growth regulators=methoprene, pyriproxyfen
Amidines=amitraz, demiditraz
Semicarbazone=metaflumizone,
Spinosyns=spinosad, spinetoram
Octadepsipeptides=emodepside,
Pyrazinoisoquinolines=praziquantel,

Preferably the at least one individual dose of topical treatment contains at least one pharmaceutically active ingredient selected from antiparasitics and insecticides, selected from the group comprising fipronil, moxidectin, imidacloprid, permethrin, cyphenothrin, methoprene, etofenprox, selamectin, dinotefuran, amitraz, metaflumizone, spinosad, emodepside, pyriproxyfen, praziquantel, and pyriprole; or any combination thereof.

Preferably the at least one individual dose of topical treatment is stored in a containment until it is applied, said containment is selected from applicator containers including, but not limited to, bottles, bottles with nozzles, bottles with brushes, spray bottles, packets, tubes, syringes, and any combinations thereof.

In another aspect the invention provides a method of marketing a topical treatment for pet animals comprising the packaging of the topical treatment into individual topical dosages, each dosage suitable for topical administration to a pet animal and including in the same package a number of oral treats for a pet animal in close proximity to the individual topical treatment dosages.

By providing the pet owner with a package that contains individual doses of topically applied medications or shampoos pre-packaged with an oral taste treat that can be administered either before the topically applied treatment is given so as to relax the pet, or after the treatment is given so as to reward the pet and help it forget the negative aspects of the treatment.

In one embodiment of the current invention the package may comprise an outer package composed of cardboard or plastic, a first containing device containing a topically applied liquid medication or shampoo and a second containing device containing one or more orally administered non-medicated taste treat sufficient for a single treatment session. The outer package may be printed with a series of instructions or may contain a separate sheet of printed instructions.

In a second embodiment the package comprises an outer package composed of cardboard or plastic, a plurality of first containing devices containing topically applied liquid medications or shampoos and a plurality of second containing devices containing orally administered non-medicated taste treats. The outer package may be printed with a series of instructions or may contain a separate sheet of printed instructions.

In a third embodiment the package comprises an outer package composed of cardboard or plastic and one or more molded unitary foil-backed blister trays in which the first containing device and second containing device are simply represented by individual compartments in the blister tray. The compartments may be able to be separated from each other by means of a score-line across the plastic tray.

In yet a further embodiment the package may comprise an outer package composed of cardboard or plastic, a plurality of first containing devices containing topically applied liquid medications or shampoos and a plurality of second containing devices containing orally administered non-medicated taste treats. The first containing devices and second containing devices may then be located or held within a cardboard tray or foil backed blister.

The package itself or some part of it may be shaped to signify the animal to be treated (and to help the owner choose the appropriate package for that species). Such a shaped package is described in U.S. patent application 20070122531 by Considine and Brown, entitled “Methods for selecting a pet-appropriate product” and published on 31 May 2007, the contents of which are incorporated herein by way of reference.

Still further embodiments are set out in the search claims annexed hereto, the contents of which are also incorporated herein by way of reference.

### BRIEF DESCRIPTION OF THE DRAWINGS

The invention will now be described, by way of example only, by reference to the accompanying drawings:

FIG. 1 shows a blister pack having separate compartments one set contains a topical liquid formulation and a second set contains an oral treat suitable for a pet, so that each treat is matched with an individual dose of a liquid treatment.

FIG. 2 shows an alternative blister pack having separate compartments one set contains a topical liquid formulation and a second set contains a pair of oral treats suitable for a pet, so that each individual dose of a liquid treatment is matched with a pair of treats.

FIG. 3 shows a typical flexible container for a topical liquid formulation that would fit inside the well in the blister pack.

FIG. 4 shows a typical treat in the form of an edible tablet that would fit inside the appropriate recess in the blister pack.

FIG. 5 shows a cardboard container having a hollow for a topical liquid formulation and a separate hollow to locate the associated treats.

FIG. 6 shows a treat shaped like a bone to signify that it is for a dog.

The following description will describe the invention in relation to preferred embodiments of the invention, namely a method of treatment of pet animals and a package designed to facilitate such a treatment. The invention is in no way limited to these preferred embodiments as they are purely to exemplify the invention only and that possible variations and modifications would be readily apparent without departing from the scope of the invention.

### DETAILED DESCRIPTION OF THE INVENTION

### Example 1

In one embodiment of the current invention the package comprises an outer package composed of cardboard or plastic, a first containing device containing a topically applied liquid medication or shampoo and a second containing device containing one or more orally administered non-medicated taste treat sufficient for a single treatment session. The outer package may be printed with a series of instructions or may contain a separate sheet of printed instructions.

The first containing device may be constructed entirely of plastic or a combination of plastic and other materials such as aluminum foil. These materials will be suited to the long-term storage of a topically applied medication or shampoo. This medication will be in the form of a solvent based liquid potentially containing one or more of the following active ingredients: fipronil, moxidectin, imidacloprid, permethrin, methoprene, selamectin, dinotefuran, amitraz, metaflumizone, spinosad, emodepside, pyriproxyfen, praziquantel and pyriprole. The second containing device may also be constructed of plastic and/or foil, however in this case the construction will be suited to the long-term storage of a soft, semi-soft or solid taste treat. This taste treat could be of either artificial or animal origin.

### Example Package Contents

| Component | From | Contents |
|---|---|---|
| Outer package | Cardboard box, plastic | First containing device, |
| | bag, foil backed blister | second containing device, |
| | | instructions |
| First container | vial, tube, foil backed | a topically applied liquid or |
| | blister tray | shampoo containing at |
| | | least one medicament |
| Second container | foil pouch, sachet, or | orally administered |
| | foil backed blister tray | medicated taste treat |

| | |
|---|---|
| Instructions | Instruction to administer one or more of the taste |
| | treats to the pet before and/or after administering a |
| | non-oral medicament or shampoo to the pet. |

### Example 2

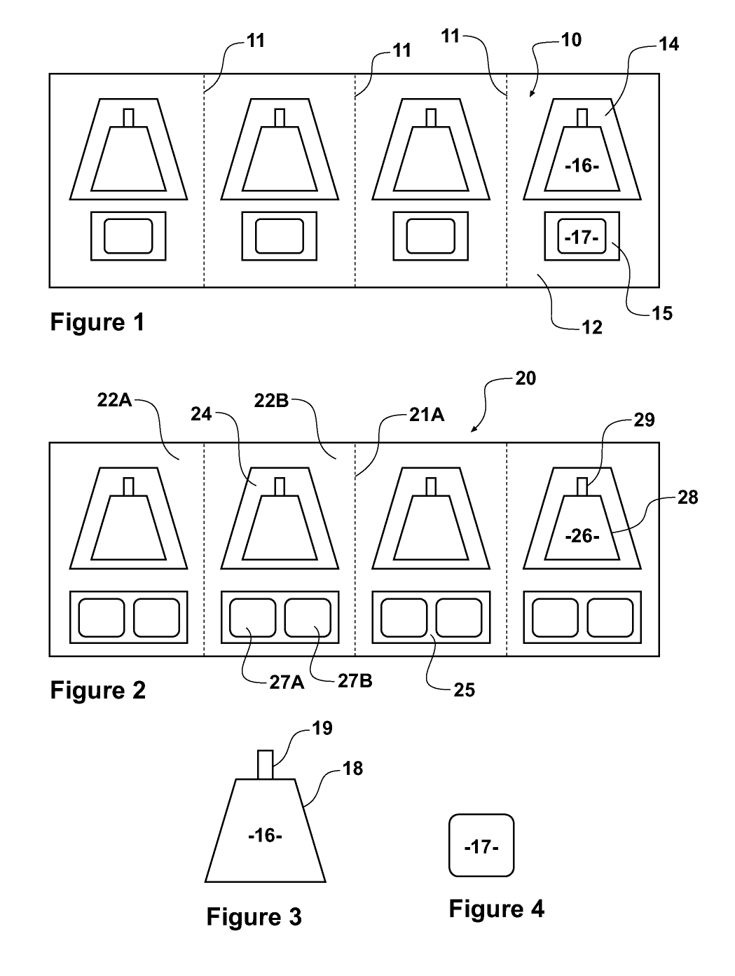

FIG. 1 shows a foil backed blister tray 10 (with foil removed to show the sets of compartments 14 and 15 and their contents 16, 17). This tray is typically sealed with foil during manufacture. Such a foil will be provided with suitable markings on the foil to show the location of the compartments, enabling the user to remove or puncture of the foil at such locations to remove both contents 16 and 17 at the same time.

The filled sealed foil tray is then stored in an outer cardboard pack until sold or used by the customer.

The tray and its foil covering have a series of perforations 11 dividing the tray into separable portions 12, each portion 12 containing one of the compartments 14 and containing one of the compartments 15.

The tray portion 12 has both a compartment 14 and a compartment 15. Compartment 14 contains a topical liquid formulation 16 in container 18. Compartment 15 contains an oral treat suitable for a pet, so that each treat is matched with an individual dose of a liquid treatment. Container 18 is shown in FIG. 3. It is preferably a squeeze bottle or pouch with a sealed nozzle 19, the bottle containing a measured amount of the topical liquid formulation.

FIG. 4 shows a typical treat in the form of an edible tablet 17, of a size to fit inside the appropriate compartment 15 in the blister pack. The type of treat is discussed in more detail below.

The cardboard package will also contain instructions on how and when to administer the treatment and when to give the treat to the pet.

In use the customer will typically remove a portion 12 by tearing along the perforation 11 and removing the foil to remove the bottle 18 and the treat 17. The user can then open the bottle by cutting the nozzle. At the same time the user can administer the treat to the pet. The user can see that by removing the foil both compartments are revealed and is reminded to give the animal the treat at the same time as the application of the topical treatment.

### Example 3

FIG. 2 shows a modified foil backed blister tray 20 (with foil removed to show the sets of compartments 24 and 25 and their contents 26, 27A, and 27B). This tray is typically sealed with foil during manufacture as previously discussed.

It has perforations 21A to separate the tray into different portions. Compartment 24 contains a bottle 28 with a nozzle 29 in a similar way to Example 2, whilst compartment 26 is large enough to contain a pair of treats 27A and 27B.

In use the customer will typically remove portions 22A, 22B by tearing along the perforation 21A and removing the foil to remove the bottle 28 and the treats 27A, and 27B. The user can then open the bottle by removing the nozzle. At the same time the user can administer a first treat 27A to the pet, and reward the pet after treatment with the second treat 27B. The user can see that by removing the foil both compartments are revealed and is reminded to give the animal the treat at the same time as the application of the topical treatment.

### Example 4

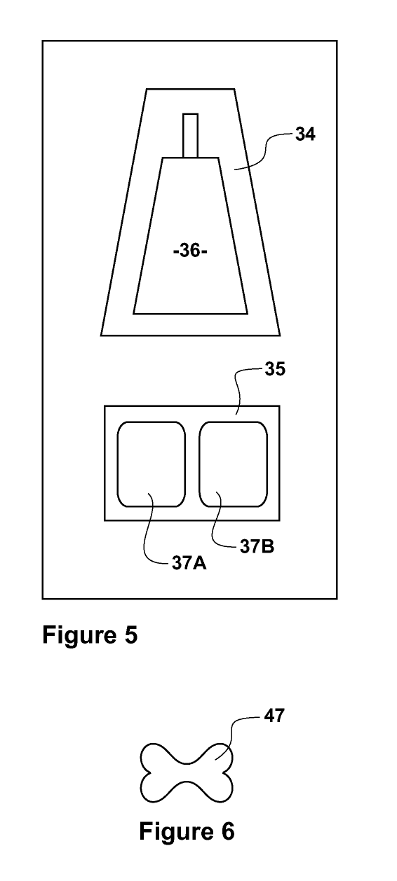

FIG. 5 shows a cardboard tray. This can be a folded tray or more preferably a moulded fibreboard tray with a suitable depression 34 to assist in locating a bottle 36. It can also have a depression or hollow 35 to locate a pair of treats 37A and 37B. This cardboard tray can be enclosed in a cardboard outer with suitable printing on the outer. If desired a transparent window can be provided on the outer to enable the customer to see the contents.

In this configuration there is typically one tray per treatment.

In use the customer will typically remove the cardboard outer (not shown) and then remove the bottle 36 and the treats 37A, and 37B. The user can then open the bottle by removing the nozzle. At the same time the user can administer a first treat 37A to the pet, and reward the pet after treatment with the second treat 37B. The user can see that by removing the cardboard outer that both compartments 34 and 35 are revealed and is reminded to give the animal the treat at the same time as the application of the topical treatment.

### Suitable Treats:

Treats can be produced according to a number of alternate methods. Among them are:
dried rawhide—in which the treat is simply a dried and cured portion of animal skin, hide or byproduct
meat pieces—dried pieces of meat such as beef jerky which may be cut into small pieces or strips
Freeze dried—pieces of meat, fish, poultry that has undergone freeze drying. These may be in the form of small pieces or flakes
Nylon chews—manufactured of nylon with flavours and odor components impregnated into the nylon substrate. These are prepared by cooking of meat and animal by-products in a pressured, steam atmosphere out of contact with a liquid phase and the volatilized flavor and odor components are condensed and absorbed in a contiguous liquid phase within the cooking vessel. This produces an impregnating solution which is free of solids and suspended liquids, such as meat particles and fat globules. The impregnation is achieved by immersing the molded nylon chew within the aqueous solution in the cooking vessel and subjecting the toy item to prolonged contact with the solution.
Extruded kibbles—in these process raw materials, such as grains, animal protein by-products etc. are ground and mixed. The mixed dry ground materials are then extruded, a process that includes mixing, kneading, proofing (rising), shaping, rising again, and slicing. The dry mix is first preconditioned to start the gelatinization of the starches. The preconditioner very accurately measures the amount of the dry mix and blends it with the measured liquid portion that can include fat, meat products, additional water, and steam. This wet mix stays in the preconditioner for about 45 seconds. While in the preconditioner, the starch is cooked about 25%. The preconditioned food then moves to an extruder, a cylindrical multi-segmented barrel that propels, mixes, and further cooks the material, and then forces it through a die where it is cut to the desired length. The product moving through the extruder produces its own friction and heat, which then cooks the mix. The speed and friction levels can be varied depending on the formula, to ensure that the product is cooked at the correct temperature for the right length of time.

The newly formed, soft and spongy ‘kibbles’ are then transferred from the extruder to the dryer where additional moisture is removed. Enrobing, the last step, entails the addition of either liquids or powders to the outer surface of the kibble. Fat and flavor enhancers are usually added at this stage.

Pressed and cut—Edible materials such as starch, rice meal, soybean, casein, denatured and partially hydrolyzed casein, protein, rawhide, dairy, meat by-products etc. are mixed with a preheated binder to form a mixture. The mixture is then fed by heated screw conveyor into a press composed of rollers. These rollers cool the material and form it into a sheet. Finally the sheet passes through a heated chamber and is cut into the desired final shapes.

Although not shown in FIGS. 1 to 5 of the drawings, these treats can be in the shape of the animal to be treated—making it easier for the owner to identify the correct packaging by the shape of the treats. See for example FIG. 6 which illustrates a bone shaped treat 47 to signify it is for a dog. Another shaped treat could be a fish shape to signify it is a treat for a cat. Or the treats could be a representation of the animal to be treated—a stylized dog as a treat for a dog, a stylized cat as a treatment for a cat. These shapes being intended to provide additional visual cues to assist the customer in purchasing the packs.

Thus various shapes can be cut or otherwise shaped from the edible material.

Baked—Meat protein and other materials is ground and mixed. When all ingredients are blended the ‘kibble’ is baked in small biscuit sized portions (plain or shaped to signify the animal species) and then if needed broken up into smaller, irregular sized pieces if a shape is not required. Baking temperatures are from 350° to 475°.

Irrespective of the form of treat they may be flavoured with natural flavours and odors, or with synthetic flavours that provide greatly enhanced storage stability. Typical flavours include Liver, Chicken, Turkey, Bacon, Fish, Beef and Lamb.

To make the treats more appealing to the pet and owner they are often cut or pre-shaped into shapes. Typical shapes include bone shaped, animal shapes, heart shapes, geometric shapes etc., each of which can be of a shape to help identify the animal species to be treated.

Treats may also either be in bite sized portions or in larger sizes designed to encourage the pet to chew and break down the piece into the bite size portions. This may have the secondary benefit of improving teeth condition.

Nylon based treats are a special category of treat in that they are designed to mimic natural bone and provide the pet with an object that provides a flavourful treat while also improving teeth condition.

More recently there has been a trend to offer treats with more natural and wholesome ingredients, with organic sourced natural proteins. Treats may be designed to contain additional vitamins, minerals, antioxidants etc. They may also contain herbal or plant based ingredients.

Treats that satisfy the need of this invention, while not being medicated according to may have such a secondary function aside from merely providing the pet with a satisfying taste sensation. The secondary function for example could be:
provide supplementary nutrients—such as antioxidants, vitamins and minerals
promote healthy skin and coat—delivery of suitable oils and other nutrients
encourage hairball removal
improve oral care—through providing a chewable material that promotes healthy teeth and gums
promote fresh breath
hip & joint

Suitable treats to satisfy the needs of this invention would preferably be of non-animal material origin and would maintain freshness and taste for the stated shelf life of the topically applied medication or shampoo with which it is packed. The advantage of making the treats of non-animal origin is that it would be easier to ship between countries and states that have concerns related to potential spread of animal diseases.

Treats satisfying the needs of this patent are those that would not be classified or regulated as Animal Drugs by the FDA or a similar governmental regulatory agency. That is the treats are not intended for use “in the diagnosis, cure, mitigation, treatment, or prevention of disease”. The treats may however fall under the classification of “dietary supplements”. That is a product intended to supplement the diet that bears or contains one or more of the following dietary ingredients:

(a) a vitamin;
(b) a mineral;
(c) an herb or other botanical;
(d) an amino acid;
(e) a dietary substance for use to supplement the diet by increasing the total dietary intake; or
(f) a concentrate, metabolite, constituent, extract, or combination of any ingredient described in clause (a), (b), (c), (d), or (e);

### INDUSTRIAL APPLICATION

The invention provides a manufactured package containing a topical liquid formulation and a treat for administration to a pet.

### ADVANTAGES OF THE PREFERRED EMBODIMENTS

The packages of the preferred embodiments are designed to help ameliorate some of the negative emotional aspects of a pet treatment occasion by providing the pet owner with a ready to use taste treat they can offer to the pet immediately before or after the treatment is applied. This would have the benefits to the pet owner of reducing feelings of guilt at having made the pet ‘suffer’. The benefit to the pet would be to make them think more positively about the treatment occasion and the fact that the owner is or has administered the treatment. The blister packs are designed to ensure that the pet owner can readily see that both the topical liquid and the treat have been administered in accordance with the instructions.

### Variations

Throughout the description of this specification, the word “comprise” and variations of that word such as “comprising” and “comprises”, are not intended to exclude other additives, components, integers or steps.

Whilst it is possible to provide separate blister packs—one containing the measured doses of the topical liquid in squeeze bottles and the other containing the treats, inside the same cardboard outer, it is not as advantageous as the link between the liquid dose and the treat may be lost, or hard to maintain.

It will of course be realised that while the foregoing has been given by way of illustrative example of this invention, all such and other modifications and variations thereto as would be apparent to persons skilled in the art are deemed to fall within the broad scope and ambit of this invention as is hereinbefore described.

1. A veterinary kit containing at least one individual dose of a topical treatment and at least one orally administered taste treat, with said topical treatment comprising a liquid spot-on treatment for cats or dogs administered for the purpose of ectoparasite and/or endoparasite control, and in which the topical treatment contains at least one pharmaceutically active ingredient selected from the group of chemical families comprising phenylpyrazoles, macrocyclic lactones, neonicotinoids, synthetic pyrethroids, insect growth regulators, amidines, semicarbazones, spinosyns, octadepsipeptides, and pyrazinoisoquinolines; or any combination thereof.

2. The veterinary kit as set forth in claim 1 wherein the at least one individual dose of topical treatment contains at least one pharmaceutically active ingredient selected from antiparasitics and insecticides, selected from the group comprising fipronil, moxidectin, imidacloprid, permethrin, cyphenothrin, methoprene, etofenprox, selamectin, dinotefuran, amitraz, metaflumizone, spinosad, emodepside, pyriproxyfen, praziquantel, and pyriprole; or any combination thereof.

3. The veterinary kit as set forth in claim 1 wherein the at least one individual dose of topical treatment is stored in a containment until it is applied, said containment is selected from applicator containers including, but not limited to, bottles, bottles with nozzles, bottles with brushes, spray bottles, packets, tubes, syringes, and any combinations thereof.

4. The veterinary kit as set forth in claim 1 wherein the kit further comprises packaging, such as, but not limited to, a blister pack, that has a first set of compartments for receiving/holding a plurality of topical treatment containers, as well as, a second set of compartments for storing a plurality of orally administered taste treats.

5. The veterinary kit as set forth in claim 1 wherein the kit contains a set whole number ratio of orally administered taste treats to individual topical treatment doses that are at least of a ratio of one to one.

6. A method of co-administering to a pet animal a topical treatment and an oral taste treat using a veterinary kit containing at least one individual dose of a topical treatment pre-packaged with at least one orally administered taste treat, said method is performed by a pet owner, veterinarian, caretaker or animal keeper to increase the acceptance/compliance of the topical treatment by using the taste treat to help relax, distract, and/or reward the animal.

7. A package for the treatment of a pet comprising:
(a) a first containing device containing a topically applied liquid,
(b) a second containing device containing an orally administered non-medicated soft, semi-soft or solid taste treat.

8. A package as claimed in claim 7, in which the topically applied liquid contains one or more medicaments in a solution or suspension applied to one or more spots on the back of the pet.
